(19) United States
(12) Patent Application Publication
(10) Pub. No.: US 2017/0265836 A1
(43) Pub. Date: Sep. 21, 2017

(54) COMPUTED TOMOGRAPHY RADIATION DOSE CHECKER

(71) Applicant: Medic Vision Imaging Solutions Ltd., Tirat Carmel (IL)

(72) Inventors: Dan LAOR, Haifa (IL); Eliran DAHAN, Haifa (IL); Eyal AHARON, Kiryat Tivon (IL); Shai ATTIA, Shimshit (IL)

(21) Appl. No.: 15/600,506

(22) Filed: May 19, 2017

Related U.S. Application Data
(63) Continuation-in-part of application No. 15/209,083, filed on Jul. 13, 2016, now Pat. No. 9,693,747.
(60) Provisional application No. 62/192,605, filed on Jul. 15, 2015.

(51) Int. Cl. A61B 6/00; A61B 6/03

(52) U.S. Cl. CPC A61B 6/582; A61B 6/032; A61B 6/54; A61B 6/461

(57) ABSTRACT

The present disclosure relates to devices and methods for embedding a standard dose checker feature within existing CT systems by obtaining and analyzing information from the existing CT system, detecting a radiation parameter value therefrom, comparing the detected radiation parameter with a predetermined threshold, and generating an operation-signal to affect the operation of the CT system based on the comparison between the detected radiation parameter and the predetermined threshold and the state of the CT system.

### TECHNICAL FIELD

The present disclosure generally relates to the field of Computed Tomography (CT) systems and uses thereof.

### BACKGROUND

During CT scanning, target areas are subject to ionizing radiation to obtain multiple X-Ray images that are then computer-processed to be combined and produce cross-sectional topographic images of the target areas in the body of the subject. The dose of the ionizing radiation in CT is typically hundreds of times higher than the dose used in conventional X-ray imaging. It is well known that high dose of ionizing radiation can be harmful to the body.

To mitigate the risk or undesired high dose exposure to ionizing radiation, regulatory entities impose certain limitations and requirements on the operation of CT scanning machines. One example of these limitations and requirements exists in a standard named XR-29, which, among other requirements, requires embedding a “dose check feature” in the CT systems to prevent operating the CT machine at a dose higher than a determined threshold, unless explicit waver/permission is provided.

This requirement is being embedded in new CT systems, while older CT systems are left without it, and, therefore, do not meet the XR-29 Standard requirements.

There is thus a need in the art for devices and methods for embedding the “dose check feature” in existing CT systems not equipped with the manufacturer's embedded Dose Check feature.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

CT systems are quite expensive medical equipment, and their cost ranges from $200K to over $500K. Consequently, many medical care centers and providers of medical imaging services, which have already obtained a CT system, may not find it affordable to upgrade to new systems that meet the XR-29 Standard requirement.

According to some embodiments, there are provided herein devices, systems and methods for implementing/embedding the “dose check feature” in existing CT systems (aftermarket). Advantageously, embedding the “dose check feature” within existing CT systems provides compliance with state of the art standards without requiring medical care centers and providers of medical imaging services to purchase new CT systems.

According to some embodiments, a dose checker-device is introduced, including an input connector (input port) configured to be connected to a CT system in a non-intrusive manner, for example, through a video link within the CT system, a processing circuitry configured to detect a radiation parameter in the CT system, and a control unit configured to affect the operation of the CT system based on the detected radiation parameter.

The terms “dose checker device”, “checker-device”, “checker device”, “checker”, “CT checker” and “SafeCT-29”, as used herein, are interchangeable.

The term “video link” as used herein refers to any link capable of transmitting imagery information to processors, monitors or other display devices.

According to some embodiment, there is provided a Computed Tomography (CT) checker device, comprising:
an input port configured to be associated with a CT-system, obtain scanning data and provide corresponding signal;
a processing circuitry, configured to:
   obtain the scanning data from the input port;
   detect a radiation parameter value from the scanning data;
   compare the detected radiation parameter value with a predetermined threshold; and
   generate an operation-signal based on the comparison,
   and
a control unit, configured to obtain the operation signal and affect an operation of the CT system based thereon.

According to some embodiment, the input port is configured to be associated with a display interface in the CT-system, wherein the scanning data is display imagery and wherein the signal is an internal-display signal, such that, the processing circuitry is configured to:
obtain the internal-display-signal from the input port;
analyze imagery depicted by the internal-display-signal;
detect the radiation parameter value from the analyzed imagery;
compare the detected radiation parameter value with a predetermined threshold; and
generate the operation-signal based on said comparison.

According to some embodiment, the terms “internal-display-signal” and “internal display signal” are interchangeable and may refer to an image, a screenshot and/or data representing an image.

According to some embodiments, the input port may be a video splitter. According to some embodiments, the input port may be a camera.

According to some embodiments, the radiation parameter includes a radiation dose.

The terms “established threshold” and “predetermined threshold” as used herein are interchangeable and refer to a radiation dose threshold. According to some embodiments, the threshold is based on an established, pre-defined, reference radiation dose level(s).

According to some embodiments, said control unit is configured to be connected to a control switch within the CT system and affect an operation of the CT system by toggling the state of the control switch thereby preventing radiation based on the comparison between the detected radiation parameter value and the predetermined threshold.

According to some embodiments, said affecting the operation comprises prevents initiation of scanning.

It is to be understood, that the device does not affect the CT system during operation, i.e., when scanning is performed. In contrast, the device includes safety means, such as, Interlock Override switch, that prevents its operation during scan acquisition.

According to some embodiments, the device further comprises a monitor, and said processing circuitry is further configured to provide a display signal to said monitor to indicate a state of operation of the device.

According to some embodiments, the state of operation of the device includes the detected radiation parameter value. According to some embodiments, the state of operation of the device includes a result of the comparison between the detected radiation parameter value and the predetermined threshold.

According to some embodiments, said processing circuitry is configured to provide a warning imagery to said display based on the comparison between the detected radiation parameter value and the predetermined threshold.

According to some embodiments, said device further comprises an output port configured to provide a display imagery signal to a monitor in the CT system, wherein the device is configured to be connected on the display link of the CT system and either pass an uninterrupted imagery from the input port to the output port, or provide an interrupted imagery from the input port to the output port based on the comparison between the detected radiation parameter value and the predetermined threshold.

According to some embodiments, said device further comprises an interface configured to obtain control-input from a user and affect the operation of the device accordingly.

According to some embodiments, said device is configured to operate a multi-level check, in which the radiation parameter is compared with a plurality of thresholds.

According to some embodiments, said detecting a radiation parameter value from the analyzed imagery comprises performing an optical character recognition on the analyzed imagery.

According to some embodiments, there is provided a method for CT dose optimization and management, the method comprising:
obtaining an internal signal from a CT system;
detecting a radiation parameter value from the internal signal;
comparing the detected radiation parameter value with a predetermined threshold; and
generating an operation-signal based on the comparison between the detected radiation parameter value and the predetermined threshold,
wherein the operation-signal is configured to affect an operation of the CT system.

According to some embodiments, the internal signal from the CT system is an internal-display-signal, wherein said method further comprises analyzing imagery depicted by the internal-display-signal, such that the radiation parameter value is detected from the analyzed imagery.

According to some embodiments, the method further comprises monitoring the operation of the CT system, and preventing the operation-signal from affecting an operation of the CT system if the CT system is in the midst of scanning.

According to some embodiments, said detecting the radiation parameter value from the analyzed imagery comprises performing an optical character recognition on the analyzed imagery.

According to some embodiments, said detecting a radiation parameter value from the analyzed imagery comprises identifying a term referring to the radiation parameter, and detecting a numerical value associated with the identified term.

According to some embodiments, the method further comprises toggling a control switch within the CT system based on the operation-signal.

According to some embodiments, said toggling a control switch comprises preventing initiation of scanning at the CT system.

According to some embodiments, said CT system is having a door switch loop configured for preventing initiation of scanning at the CT system and said control switch is connected to the door switch loop.

According to some embodiments, said control switch is configured to prevent interruption of the CT operation if CT scanning is being performed.

According to some embodiments, said control switch may be overridden to prevent interruption of the CT operation in case of a failure in the Dose checker device.

According to some embodiments, there is provided a method for CT dose optimization and management, the method comprising:
providing a CT checker device, comprising
   an input port, configured to be associated with a CT-system, obtain scanning data therefrom, and provide an internal signal;
   a processing circuitry, configured to:
      obtain the internal signal from the input port;
      detect a radiation parameter value from the internal signal;
      compare the detected radiation parameter value with a predetermined threshold; and
      generate an operation-signal based on the comparison,
      and
   a control unit, configured to obtain the operation signal and affect an operation of the CT system based thereon,
obtaining an internal signal from a CT system;
detecting a radiation parameter value from the internal signal;
comparing the detected radiation parameter value with a predetermined threshold; and
generating an operation-signal based on the comparison between the detected radiation parameter value and the predetermined threshold,

wherein the operation-signal is configured to affect an operation of the CT system.

According to some embodiments, said input port is configured to be connected to a display interface in a CT-system, said scanning data is display imagery and said internal signal is an internal-display-signal.

According to some embodiments, there is provided a method of identifying and calibrating CT model display layout, comprising:
providing a database of CT models comprising a plurality of CT models each associated with at least one unique element and at least one screen layout comprising at least screen elements, a video splitter configured to capture images of a CT operator display of an existing CT system, and a video grabber,
connecting the video splitter and the video grabber to the CT operator display,
capturing at least one image of the CT operator display;
transmitting the at least one captured image to the video grabber;
defining a first search window for scanning throughout the captured image;
identifying a CT model by comparing the contents of the first search window with the at least one unique element in each CT model within the database of CT models;
retrieving the at least one screen element associated with the identified CT model;
generating a screen layout comprising the at least one screen element; and
calibrating and optimizing scan parameters for each screen element.

According to some embodiments, the database further comprises at least one software version for at least one CT model.

According to some embodiments, the identifying a CT model further comprises identification of the software version of the CT model.

According to some embodiments, the method further comprising changing the size of the first search window if a unique element was not identified.

According to some embodiments, the generating a screen layout further comprises:
defining a second search window for scanning throughout the captured image;
comparing contents of the second search windows with screen elements;
validating values of the screen elements; and
verifying that all screen elements comply with preset rules.

According to some embodiments, the method further comprises changing the size of the second search window if a unique element was not identified.

According to some embodiments, the calibrating and optimizing scan parameters comprises:
defining a range of zoom levels;
defining a range of threshold function;
executing OCR functionality for each zoom level in the range of zoom levels and for each threshold function for the range of threshold functions;
identifying a correct value as the most common value of the series of OCR executions; and
identifying the fastest OCR execution time for all correct values.

According to some embodiments, the calibrating and optimizing scan parameters is executed for all screen elements.
According to some embodiments, the method further comprises: storing data comprising: x coordinate, y coordinate, width, height, zoom level, threshold function, OCR execution time and correct value, for all screen parameters, and
presenting a graphic representation of the stored data.

According to some embodiments, the method further comprises performing manual quality control.

According to some embodiments, the performing manual quality control comprises manual approval of the presented stored data.

According to some embodiments, the performing manual quality control comprises manual update of a portion of stored data, comprising: x coordinate, y coordinate, width, height, and zoom level.

According to some embodiments, there is provided a method of calibrating and optimizing scan parameters, comprising:
providing a scanned image;
identifying screen elements in the scanned image;
defining a range of zoom levels;
defining a range of threshold functions;
executing OCR functionality for each zoom level in the range of zoom levels and for each threshold function for the range of threshold functions;
identifying a correct value as the most common value of the series of OCR executions; and
identifying the fastest OCR execution time for all correct values.

According to some embodiments, there is provided a method of identifying and calibrating CT model display layout, comprising:
providing a database of CT models comprising a plurality of CT models each associated with at least one unique element and at least one screen layout comprising a list of screen elements, a video splitter configured to capture images from a CT operator display of an existing CT-system, a video grabber, and a CT checker device, the CT checker device comprising:
   an input port configured to be associated with the existing CT-system, the input port configured to obtain scanning data and provide corresponding signal;
   processing circuitry; and
   a control unit, connected to a control switch within the existing CT system,
connecting the video splitter and the video grabber to the CT operator display;
capturing at least one image of the CT operator display;
transmitting the at least one captured image to the video grabber;
defining a first search window for scanning throughout the captured image;
identifying a CT model by comparing the contents of the first search window with the at least one unique element in each CT model within the database of CT models;
retrieving the at least one screen element associated with the identified CT model; and
generating a screen layout comprising the at least one screen element; and
calibrating and optimizing scan parameters for each screen element,
wherein the processing circuitry is configured to obtain the screen elements; detect a radiation parameter value from the scanning data; compare the detected radiation parameter value with a predetermined threshold; and generate an operation-signal based on the comparison,
and wherein the control unit is configured affect an operation of the existing CT system, based on the operation-signal, by toggling the state of the control switch thereby preventing radiation, wherein the checker is separate from the existing CT system.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a common CT system to monitor connectivity;

FIG. 2 schematically illustrates a system with a checker connected between the CT system and the CT monitor, according to some embodiments;

FIG. 3 schematically illustrates a CT checker with CT monitor control, according to some embodiments;

FIG. 4 schematically illustrates a system with a CT checker not intervening with the CT monitor link, according to some embodiments;

FIG. 5 schematically illustrates a CT checker without a CT monitor control, according to some embodiments;

FIG. 6 illustrates a flow chart of a method of operating a checker, according to some embodiments;

FIG. 7A schematically illustrates a setting with a SafeCT-29 checker, according to some embodiments;

FIG. 7B schematically illustrates a setting with a SafeCT-29 checker, according to some embodiments;

FIG. 8 schematically illustrates a setting with a SafeCT-29 checker, according to some embodiments;

FIG. 9 schematically illustrates a setting with a SafeCT-29 checker, according to some embodiments;

FIG. 10 schematically illustrates a setting with a SafeCT-29 checker, according to some embodiments;

FIG. 11 schematically illustrates a setting with a SafeCT-29 checker, according to some embodiments;

FIG. 12 illustrates an exemplary dose notification, according to some embodiments;

FIG. 13 illustrates an exemplary dose alert, according to some embodiments;

FIG. 14a illustrates an exemplary screen shot adapted from CT Operator display, according to some embodiments;

FIG. 14b illustrates an exemplary screen shot adapted from CT Operator display, according to some embodiments;

FIG. 15 illustrates an exemplary screen layout with graphic elements, according to some embodiments;

FIG. 16 illustrates a flow chart of a method for CT model layout identification and calibration, according to some embodiments;

FIG. 17 illustrates a flow chart of a method for CT scanner model and software version identification, according to some embodiments;

FIG. 18 illustrates a flow chart of a method for screen layout identification, according to some embodiments; and

FIG. 19 illustrates an exemplary calibration and optimization User Interface layout, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there are provided herein add-on devices to existing CT systems, systems and methods for implementing the “dose check feature” in existing commercial CT systems. Advantageously, embedding the “dose check feature” within existing CT systems provide numerous advantages, including, but not limited to:
(i) provides compliance with state of the art standards without requiring medical care centers to purchase new CT systems and supports specified legally marketed CT scanners of any vendor presenting estimated dose on the CT operator's display prior to the scan;
(ii) designed to operate in the various display protocols of any scanner and any CT scanner software versions, which display the dose information in different formats and locations on the CT console display;
(iii) includes a configuration file, which defined the various scanners known in the art and their corresponding software versions, together with the related information on the location of the relevant information on the CT display and the scanner's workflow. The configuration file is updated occasionally, such that there relevant details of every new CT scanner model that is introduced to the market are added thereto. An exemplary list of scanners and the corresponding Software (SW) versions included in a given configuration file is provided in Table 1.

TABLE 1
An exemplary configuration file

| Manufacturer | CT Model | SW/OS Version |
|---|---|---|
| GE | Discovery LightSpeed | OS SUN OS 5.8 |
| GE | Discovery RX | dm09_dvctsp1.23 |
| GE | Discovery ST | dm09_hl2sp1.23 |
| GE | LightSpeed Plus | 308-2_H3.1M5 |
| GE | LightSpeed Pro 16 | 07MW11.10 |
| GE | LightSpeed VCT | gmp_vct.42 |
| Philips | Brilliance 64 | 3.5.5 |
| Philips | Brilliance 64 | 2.6.2 |
| Philips | GeminiGXL 16 | 2.2.5 |
| Toshiba | Acquilion | V3.35ER007 |

(iv) does not alter the integrity of the CT system, thus, post implementation of the dose check feature all the functions of the CT scanner are preserved. Moreover, dose check feature implementation does not affect the design nor operation of the scanner (i.e. none of the CT controls such as X-ray, motion, GUI are affected). This advantage is conferred by the fact that the entire dose check feature system, including the software, is separated from the CT scanner. In fact, the dose check feature software runs on an independent computer and no 3rd party software runs on the CT Console (or any other part of the scanner);
(v) interfaces with the CT system through standard connections;
(vi) continuously receives the CT Console display video: the CT protocol and radiation dose information (calculated by the scanner) that are displayed to the CT operator may be extracted and analyzed in real time by the checker-device software. To this end, the checker-device software may include certain measures aimed to ensure correct reading of the data items. Thus, the checker-device presents dose notifications and prevents over-dose scanning in a timely manner, that is, it does not cause delays in operating the CT scanner. According to some embodiment, the checker-device performs its required functionality within less than 300 ms, i.e. from the time a certain data item (e.g. dose level) appears on the screen until the required action is performed (e.g. display a notification message).

According to some embodiment, the checker-device software includes a watchdog mechanism in order to protect from software or hardware failures that may cause the system to stop responding.

According to some embodiments, an add-on checker-device is introduced to an existing commercial CT system, including an input connector, configured to be associated with a CT system, a processing circuitry configured to detect a radiation parameter in the CT system, and a control unit configured to affect the operation of the CT system based on the detected radiation parameter.

According to some embodiments, the input connector (also termed ‘input port’) is a remote input connector, and is not physically linked to the CT system.

According to some embodiments, the input port is connected to the CT system.

According to some embodiments, the radiation parameter includes a radiation dose.

According to some embodiment, the radiation dose is expressed in terms of CTDIᵥₒₗ, optionally, in units of milliGrays or in CT power distribution unit (CT PDU).

According to some embodiments, the radiation parameter includes radiation intensity. According to some embodiments, the radiation parameter includes radiation frequency. According to some embodiments, the radiation parameter includes radiation amplitude at various frequencies. According to some embodiments the radiation parameter includes radiation duration. According to some embodiments, the radiation parameter includes any combination of the above.

According to some embodiments, the radiation parameters are compared to an established threshold (predetermined threshold or predefined threshold).

According to some embodiments, the radiation parameters are compared to a plurality of thresholds. According to some embodiments, the processing circuitry configured and/or control unit are configured to affect the operation of the CT system based on the comparison between the radiation parameter(s) and the thresholds.

According to some embodiments, affecting the operation may include presenting a warning message. According to some embodiments, affecting the operation may include sounding and/or producing an alarm. According to some embodiments, affecting the operation may include obstructing radiation. According to some embodiments, affecting the operation may include switching a safety switch (toggle).

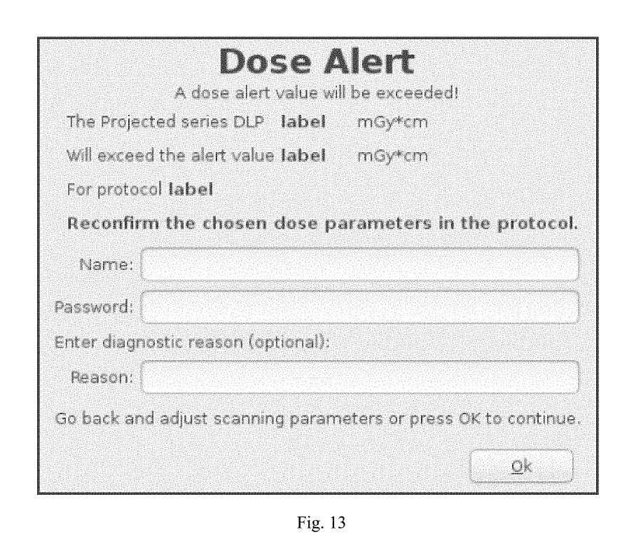

The terms “warning message”, “warning imagery”, “alarm”, “alarm message” and “alarm imagery”, as used herein, are interchangeable and may refer to a message as exemplified in FIGS. 12 and 13.

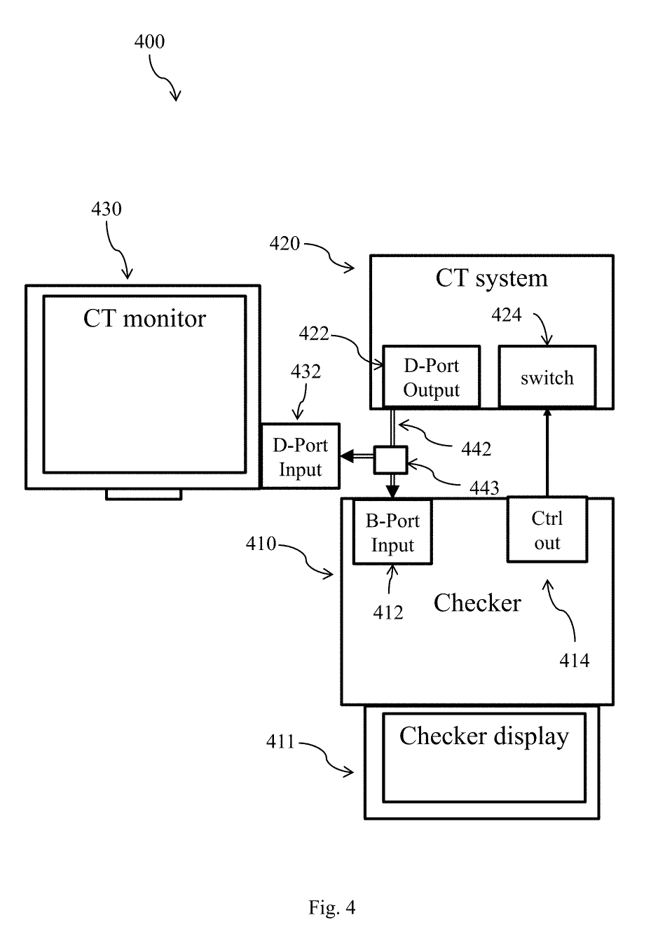

The terms “control switch”, “safety switch” and “toggle”, as used herein, are interchangeable and may refer to a switch as exemplified in FIG. 4.

According to some embodiment, checker-device interfaces with the CT system through standard connections in accordance with the CT manufacturer recommendations.

According to some embodiment, add-on checker-device interfaces with the existing CT system through a connection selected from the group consisting of: (1) the CT Console video output, (ii) the CT door Switch loop, and (iii) the X-ray warning light circuit.

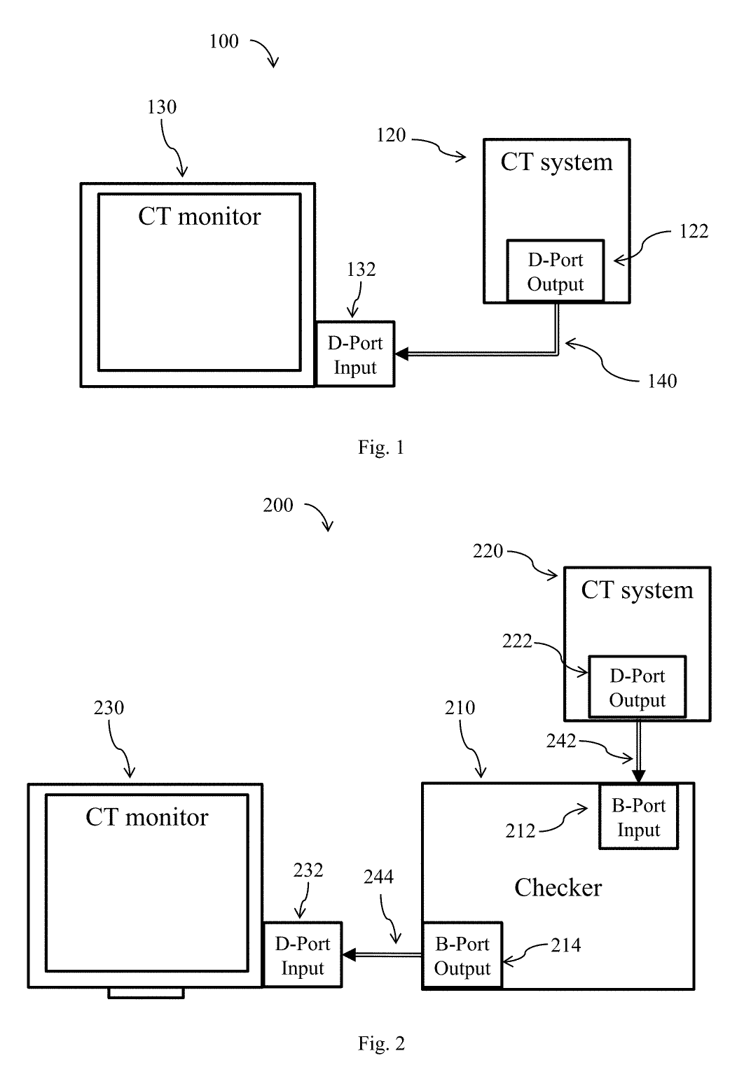

Reference is now made to FIG. 1, which schematically illustrates a common setting 100 of a CT system 120 to CT monitor 130 connectivity. According to some embodiments, CT system 120 is commonly connected to CT monitor 130 by a display link 140 connected to an output display port 122 in CT system 120 and an input display port 132 in CT monitor 130.

Display link 140 and display input and output ports 132 and 122 may include standard display connections such as Composite video, SCART, S-Video, CGA, MDA, HCG, EGA, Amiga video, VGA, GVIF, OpenLDI, DVI, SDI, HDMI, DisplayPort, DiiVA, HDBaseT, CoaXPress, MHL, or the like.

According to some embodiments, the add-on CT checker is configured to obtain display signal(s) from the display link, analyze an image depicted by the signal(s), detect a radiation parameter, and compare the detected parameter with a predefined threshold or range of values. According to some embodiments, the device is further connected to a control switch to affect the operation of the CT system based on the comparison.

Reference is now made to FIG. 2, which schematically illustrates a setting 200 with add-on CT checker 210 connected between the existing CT system 220 and the existing CT monitor 230, according to some embodiments. According to some embodiments, CT system 220 generates display information and provides it through an output port 222 to a display link 242. CT checker 210 is connected to display link 242, and configured to analyze the imagery depicted therein to detect a value of a radiation parameter.

Then CT checker 210 either provides the same image uninterruptedly through a checker output display port 214 via a checker display link 244 to a monitor input port 232 to be displayed on CT monitor 230, or interferes with the image, for example by introducing warning messages, based on the value of the detected parameter.

The terms “add-on CT checker”, “add-on checker device”, “checker device” and “CT checker”, as used herein, are interchangeable and refer to an external add-on device of the current disclosed technology.

The terms “existing CT system” and “CT system”, as used herein, are interchangeable, and refer to a commercial CT system to which an external add-on CT checker, according to the current disclosed technology, connects.

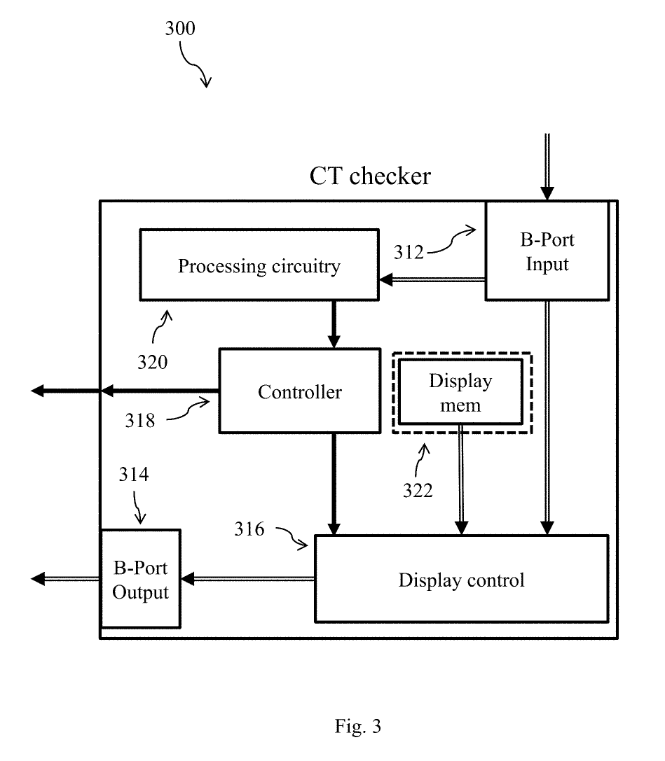

Reference is now made to FIG. 3, which schematically illustrates a CT checker 300 with CT monitor control, according to some embodiments. According to some embodiments, CT checker 300 includes a checker input display port 312 configured to obtain a display signal from a CT system, and provide the signal to a display controller 316 and a processing circuitry 320. According to some embodiments, processing circuitry 320 is configured to analyze the provided imagery, and detect a radiation parameter, then to compare it with a predetermined threshold value or range, and accordingly instruct a controller 318 to interfere with the operation of the CT system, and/or configure display controller 316 to affect a change on an image provided to a CT monitor through a checker output display port 314. According to some embodiments, CT checker 300 may further include a display memory 322 having stored thereon predefined imagery to be displayed as requested by controller 318.

According to some embodiments, the predefined imagery may be a warning signal and or a notification. According to some embodiments, the warning signal may include a warning text.

According to some embodiments, the CT checker is configured to be connected to the display link of the CT system, without interrupting the signal provided to the CT monitor.

Reference is now made to FIG. 4, which schematically illustrates a setting 400 with a CT checker 410 not intervening with a CT monitor link 442, according to some embodiments. According to some embodiments, CT checker 410 is configured to obtain a display signal through a checker input display port 412, the signal depicting imagery data provided by a CT system 420 through an output display port 422 therein, to a CT monitor 430 through an input display port 432 therein.

According to some embodiments, CT checker 410 is configured to analyze the imagery, detect a parameter value therefrom, and compare the value with a predefined threshold or range, and, based on the comparison criteria, provide a control signal to CT system 420 via a control output 414, for example, to control an operation safety switch 424 in CT system 420.

According to some embodiments, said safety switch 424 is the door switch loop of the CT system.

According to some embodiments, CT Checker 410 may further include a checker display 411 configured to display information related to the checker comparison status, detected parameter, and the like.

According to some embodiments, setting 400 includes a video splitter 443, configured to obtain the video signal from CT system 420 and split it to be provided to CT monitor 430 and checker 410.

According to some embodiments, the Video splitter is configured to capture images from the CT monitor (CT Operator display video) and sends a copy of same video/image signal to a Video Grabber. This function may be performed continuously and automatically (such that the video splitter can be always “on” with no requirement for user action or interface). According to some embodiments, The Video Splitter is a passive device that takes the output signal out of the CT console and splits that into two identical output signals: one configured to be provided to the CT system (CT console) and the other goes to the checker (SafeCT-29 system).

According to some embodiments, a checker processing circuitry (SafeCT-29 Computer) may include an Off-The-Shelf (OTS), high quality video splitter that ensures that the quality of the image displayed on the CT console is maintained without significant or any image quality degradation.

According to some embodiments, technical characteristics of the SafeCT-29 Video Splitter may include: 2 Port Internal Video Splitter, Video Input: 15-pin HD-15 connector, Video Output: 2×15-pin, HD-15 connectors, Bandwidth of 250 MHz, Supported VGA Modes: All modes up to 1920×1440, Power: 5V 200 mA, Fault tolerant output port, Integrated ground loop isolation, Low voltage circuitry and OS Support: Linux.

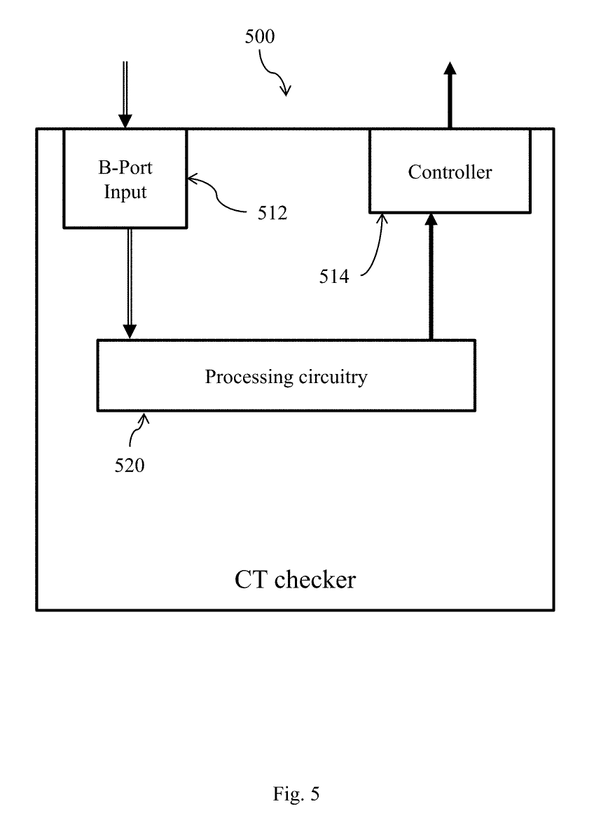

Reference is now made to FIG. 5, which schematically illustrates a CT checker 500 without CT monitor control, according to some embodiments. According to some embodiments, CT checker 500 is configured to obtain a display signal through a checker input port 512, then analyze imagery depicted in the signal utilizing a processing circuitry 520 to detect a radiation parameter, and check the parameter using a defined criteria, and provide a control signal through a controller 514 based on the parameter value and the criteria.

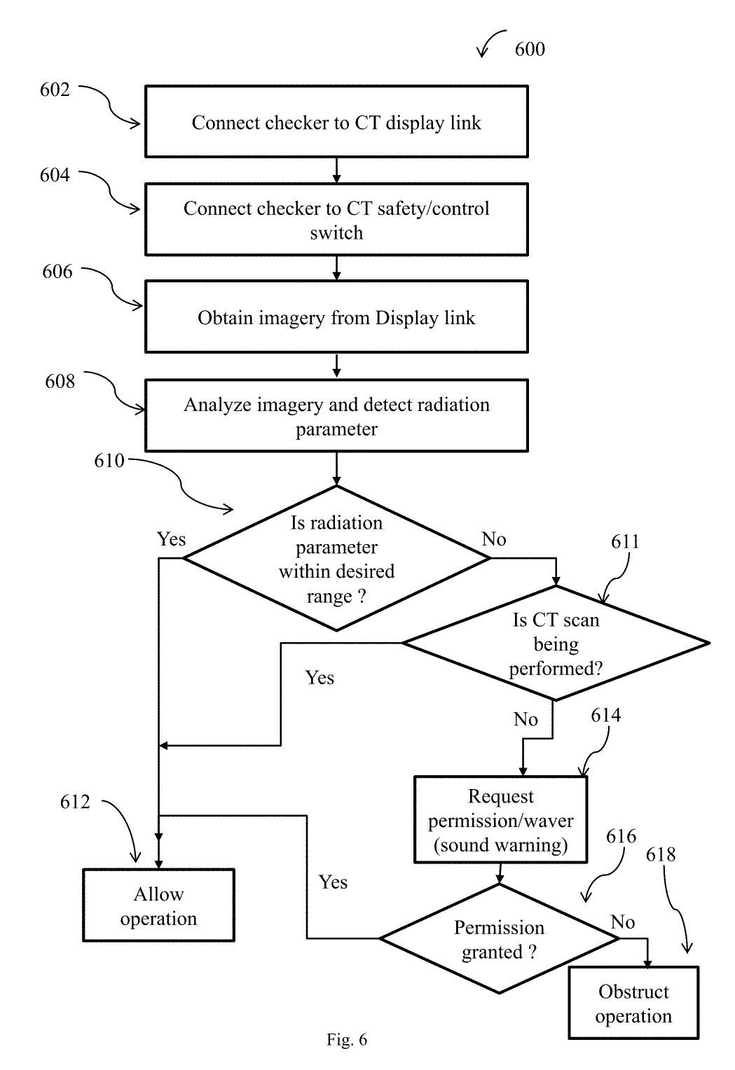

Reference is now made to FIG. 6, which illustrates a flow chart of a method 600 of operating a checker, according to some embodiments. According to some embodiments, method 600 begins by connecting the checker to a CT display link (step 602), and connect the checker to a CT safety/control switch (step 604), then obtain imagery from the display link (step 606), and analyze the imagery to detect a radiation parameter (step 608). Then, the detected parameter is compared with a threshold or a defined criteria (step 610), and if the parameter passes the criteria, CT operation is allowed (step 612), otherwise, the state of the CT system is checked to verify if a scan is already being performed (step 611). If so, the checker allows the CT system to continue scanning with no interruption. Otherwise, a permission/waver is requested (step 614), and if the permission is granted (step 616) operation may be allowed (step 612), otherwise, operation will be obstructed (step 618).

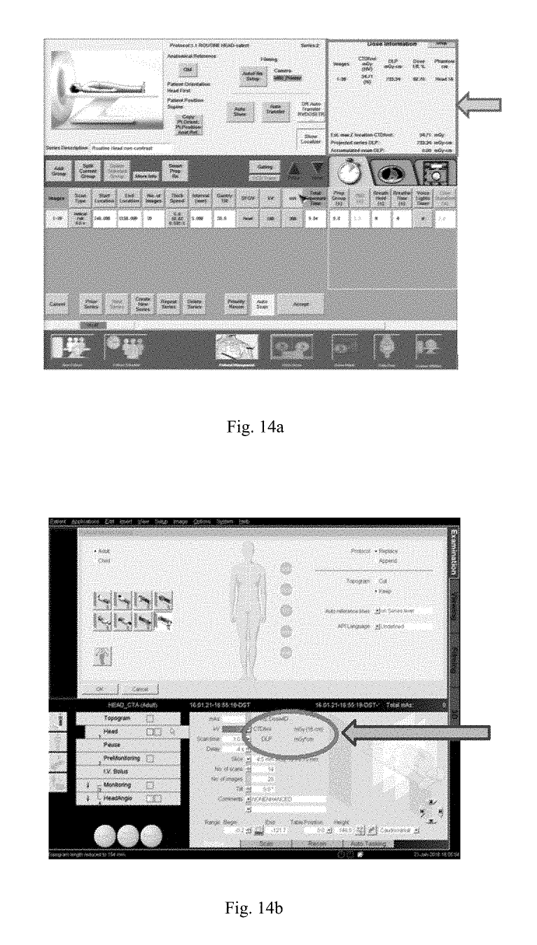

According to some embodiments, the CT Operator display video is analyzed by an integrated OCR software to detect the radiation parameter values from the analyzed imagery. It is to be understood, that the layout of data parameters, including the location of the radiation parameters on the CT Operator display (i.e. within the internal-display signal) varies from one CT model to another. Examples of layouts are provided in FIG. 14a and FIG. 14b.

FIG. 14a is an exemplary screen-shot adapted from CT Operator display used by GE Lightspeed scanner and FIG. 14b is an exemplary screen-shot adapted from CT Operator display used by Siemens Sensation scanner. It is noted that the radiation parameters, indicated by an arrow in both examples, are located in different positions for each layout. The terms “layout” and “screen layout”, as used herein, are interchangeable and refer to a list of specific graphic elements that appear on a display screen, with their related characteristics.

The terms “screen element” and “graphic element”, as used herein, are interchangeable and refer to a string of text or an icon, the string of text or icon being displayed on a display screen, and being associated with related characteristics. The characteristics may include, but are not limited to, location expressed as x coordinate and y coordinate, size expressed as height and width in pixels, and type (such as text or icon).

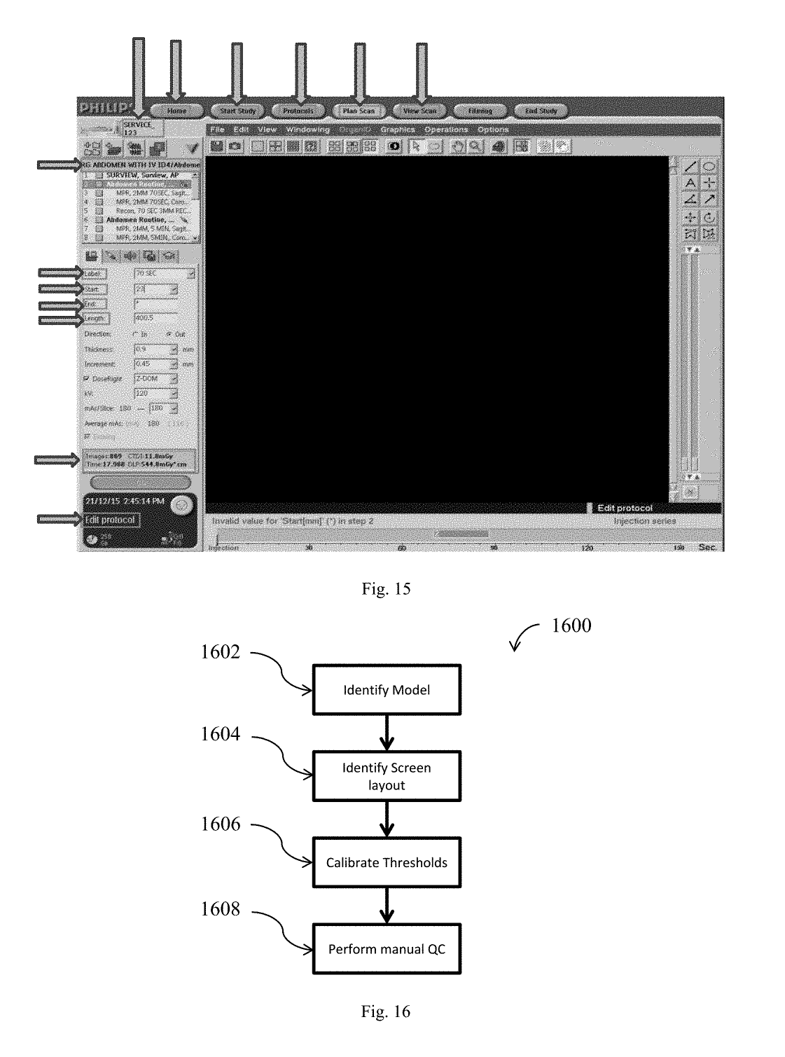

An example of a screen layout is provided in FIG. 15, in which different graphic elements are indicated by arrows. Non-limiting examples of screen elements can be: radiation parameters, CT scan characteristics, patient's name and other information related to a patient.

The term “text”, as used herein, refers to any of alphabetical characters, special characters, numbers and symbols.

The exact location, which can be expressed by (x,y) coordinates, of the radiation parameters, as well as other parameters of interest displayed in the CT Operator display, can vary from one CT model to another. Moreover, location of parameters and screen layout can vary for the same CT model between different software versions, or due to other differences between CT systems that may arise as a consequence of dissimilarities in hardware components of the CT console that generates the video signal, the display characteristics and the cables that connect the CT console to the CT Operator display. Such variations in screen layout or locations can be significant (e.g. 10 pixels), and can result in erroneous identification of the retrieved data, such as the radiation parameters. Therefore, there is a need for a method to automatically identify the model and software version of the CT scanner, and to calibrate the SafeCT-29 system to ensure proper operation of the system either during first installation, during periodical setups and re-calibrations, and during ongoing operational use of the system.

According to some embodiments, a database of CT models is defined and stored in the SafeCT-29 system memory. According to some embodiments, each CT model in the database is associated with at least one software version, and at least one screen layout, wherein each screen layout comprises at least one screen element. According to some embodiments, the database is updated periodically. According to some embodiments, the Video splitter captures the CT Operator display video and sends a copy of same video signal to the Video Grabber, wherein said captured image is compared to the database of CT models, optionally associated with specific software versions and their screen layouts.

Reference is now made to FIG. 16, which illustrates a flow chart of a method 1600 for CT model layout identification and calibration. According to some embodiments, method 1600 begins by identification of the CT model and software version (step 1602), followed by generation (also known as identification) of the screen layout (step 1604), threshold calibration (step 1606) and finally performance of a manual Quality Control (QC).

The term “unique element”, as used herein, refers to a graphical representation, textual representation, or any combination thereof, which is unique to a specific CT model or software version, and cannot be found in other CT models of software versions.

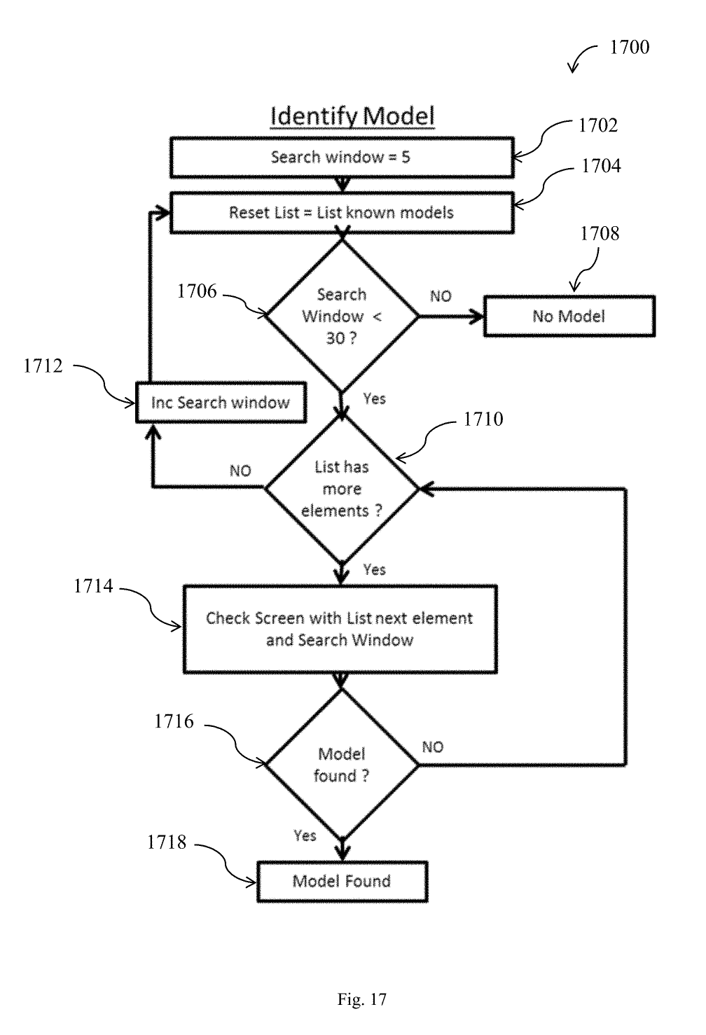

Reference is now made to FIG. 17, which illustrates a flow chart of a method 1700 (equivalent to step 1602 608 in FIG. 16) for CT scanner model and software version identification. The screen layout of each CT scanner model, and for specific software versions of each model, can include at least one unique element. According to some embodiments, the database of CT models comprises at least one unique element associated with each CT model. According to some embodiments, the database of CT models further comprises at least one unique element associated with at least one software version. According to some embodiments, method 1700 begins by defining a minimal first search window (step 1702). For example, a first search windows of 5 pixels by 5 pixels can be defined. The first search window is used to search for a unique element throughout the captured image, wherein said first search window is being advanced horizontally and vertically across all or some (x,y) locations of the captured image, each time the content of the first search window is being compared to a currently selected unique element stored in the database of CT models (step 1716). If a specific CT model or software version are not identified (step 1716), the system will look for the next unique element in the list (step 1710) and repeat the comparison process. If no elements are left in the list of unique elements, the size of the first search window is increased (step 1712), and the list of unique elements, associated with CT models or software versions, is reset (step 1704) to start the search process with the increased first search windows size for all unique elements. According to some embodiments, the first search window in step 1712 is increased by at least one pixel. A maximal first search window size is preset in the system. For example, a maximal size of 30 pixels by 30 pixels. The first search window is compared to the preset maximal window size (step 1706). If the size of the first search window exceed the preset maximal size, identification fails and the process ends without being able to identify the CT model or software version (step 1708), indicating the CT model, or software version, is new. If a unique element is identified (step 1716), the associated CT model or software version associated with the identified unique element is marked as the identified model, and the screen layout associated with identified CT model or software version is retrieved.

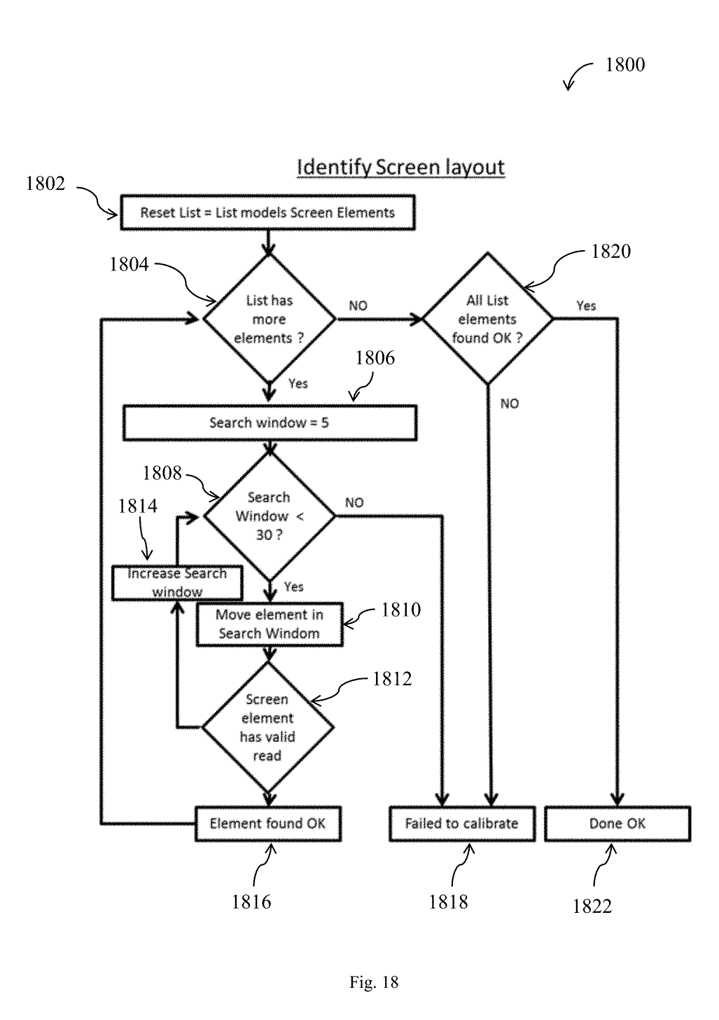

Reference is now made to FIG. 18, which illustrates a flow chart of a method 1800 (equivalent to step 1604 in FIG. 16) for screen layout generation, also termed screen layout identification. The retrieved screen layout of the identified CT model or software version comprises a list of screen elements. According to some embodiments, method 1800 begins by the retrieval of all screen elements of the identified CT model of software version (step 1802). A minimal second search window is defined (step 1806). For example, a second search windows of 5 pixels by 5 pixels can be defined. The second search window is used to search for a screen element throughout the captured image, wherein said second search window is being advanced, for example, pixel by pixel horizontally and vertically across all (x,y) locations of the captured image, each time the content of the second search window is being compared to a currently selected screen element retrieved from the list of screen elements (step 1810). According to some embodiments, the search window is advanced vertically and horizontally by any predefined amount of pixels each time, for example, 2 pixels at a time. According to some embodiments, the search window is advanced vertically and horizontally across predefined portion locations of the captured image. According to some embodiments, the database of CT models and software versions can include typical locations of screen elements, such that the second search window is advanced horizontally or vertically in the vicinity of such typical locations, i.e.—advanced within a limited range of relative to given location (x,y) coordinates. A screen element and its values are interpreted by optical character recognition (OCR) process analysis. If the screen element or its accompanying value is not valid (step 1812), the size of the second search windows is increased (step 1814). According to some embodiments, the second search window in step 1814 is increased by at least one pixel. A maximal second search window size is preset in the system. For example, a maximal size of 30 pixels by 30 pixels. The second search window is compared to the preset maximal window size (step 1808). If the size of the second search window exceed the preset maximal size, the process stops with an indication of calibration failure (step 1818). If the screen element and its accompanying value are valid (step 1812), the characteristics of the valid element are documented and stored. Valid element characteristics can include, but are not limited to, location and size. The system checks next whether there are any more elements left in the list of elements (step 1804), and if there is at least one more element in the list, the process of scanning for the element and its value is repeated. Once no more elements are left unidentified in the list of elements (step 1804), the system checks whether all elements comply with a predefined set of rules. Said rules can be that the values are within a legitimate predefined range, for example. If this condition fails, the process stops with an indication of calibration failure (step 1818). If all elements comply with the predefined rules, the process ends with an indication of successful identification of screen layout (step 1822).

The method of calibration and optimization (equivalent to step 1606 in FIG. 16), starts with a definition of a range of zoom levels and a range of threshold functions for each screen element.

The term “threshold function”, as used herein, refers to threshold value for gray levels in an image, such that the threshold function filters all gray levels that are below the threshold. A threshold spectrum can be translated to a predefined range of, for example, a scale between 1 and 200. A threshold function can be executed in steps, for example, from 1 to 100, such that for the scale of 1-200, a threshold function of 80 will filter all gray levels below 80, leaving an image with all gray level equal to and above 80 up to 200.

The term “zoom level”, as used herein, refers to a level indicative of a magnification of a screen element. For example, seven (7) zoom levels can be defined, such that each level is representative of an actual zoom. A zoom level of 7 can be translated, for example, to an actual zoom of ×4, meaning a magnification of 400% whereas a zoom level of 1 can be translated, for example, to the actual size of the element (i.e. ×1).

For each screen element, the OCR functionality is executed for each zoom level from the predefined range of zoom levels (for example, 5 zoom levels), and for each threshold function from the range of threshold functions (for example, 1 through 100). The resulting value from the OCR functionality executed for each zoom level and threshold function is stored in a list of results, as along with the associated zoom level, threshold function, and OCR execution duration. OCR execution duration can be a function of, for example, OCR run-time. At the end of OCR executions, the system identifies the most common result value within the list of results. The most common result value is then defined as the correct-value. The system then looks for the shortest execution time amongst all results that provide correct-values and defines the related parameters (zoom level and threshold function) as the “Selected Parameter Set”, also termed “scan parameters”. Selected Parameter Sets are then stored for the associated screen elements, to be used during all future CT Operator display scan readings. This process is repeated for all screen elements.

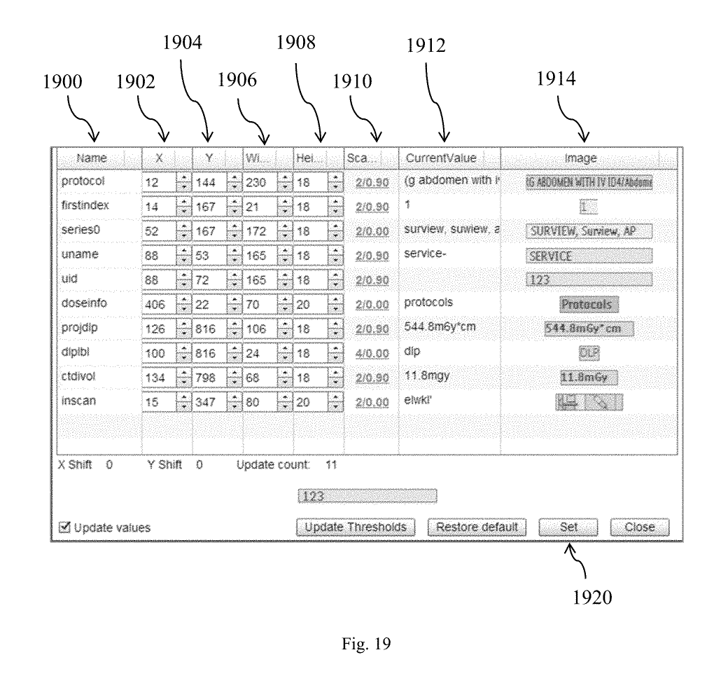

Reference is now made to FIG. 19, which illustrates an exemplary calibration and optimization User Interface (UI) layout, according to some embodiments. For all screen elements 1900 the system stores: the X coordinate 1902 and Y coordinate 1904, which can be indicative of any specific corner or the center of the location of screen element 1900, the width 1908 and height 1910 of the screen element 1900, the stored scaling parameters 1910, which can include, but are not limited to, the zoom level, the threshold function, and the OCR execution time, the correct value 1912 of the screen element 1900, and an image of the element 1914, wherein the image of the element is embedded in image format, such as, but not limited to: TIFF, BMP, GIF and JPG.

At the end of the calibration and optimization process, a system's user can perform a Quality Control (QC) by reviewing the results, which can be visualized through a Graphical User Interface (GUI) such as the exemplary layout provided in FIG. 19, and validate the results presented, for example, by clicking an approval button 1920. Alternatively, the system's user can perform a manual calibration of some of the presented characteristics, such as X coordinate 1902, Y coordinate 1904, Width 1906, Height 1908, Zoom level and threshold function 1910.

The complete process 1600 or a portion of the process, such as manual QC, can be re-performed and updated periodically or according to any requirements set by a system's user.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

### Examples

### Example 1. Video Splitter Connections

The Checker-device may include a Video Splitter aimed to prevent interference with the functionality of the CT system through the CT video connection (which may result in a poor display or absence display on the CT Operator's Display). The Video Splitter may exert its beneficial activities by operating as follows:

1. ensure no loss in picture quality, resolution or power and no additional loading limitations on the video cables.

2. has an integrated ground loop isolation circuitry eliminating all VGA noise caused by a ground loop.

3. is Fault-tolerant, providing a video output that is identical to the input even when the device fails or powered-off. The fault-tolerant output is connected to the CT Operator's display, thus ensuring proper operation of the scanner in case of checker-device failure.

4. uses a low voltage transition-minimized differential signaling circuitry to ensure that it cannot cause damage to the CT display or the video source (the CT Console).

5. when the checker-device software identifies noise, instability and/or artifacts in the video signal, or no signal, a warning message is displayed on the checker-device Display, to alert the CT Operator of such display noise.

### Example 2. System Architecture and Components

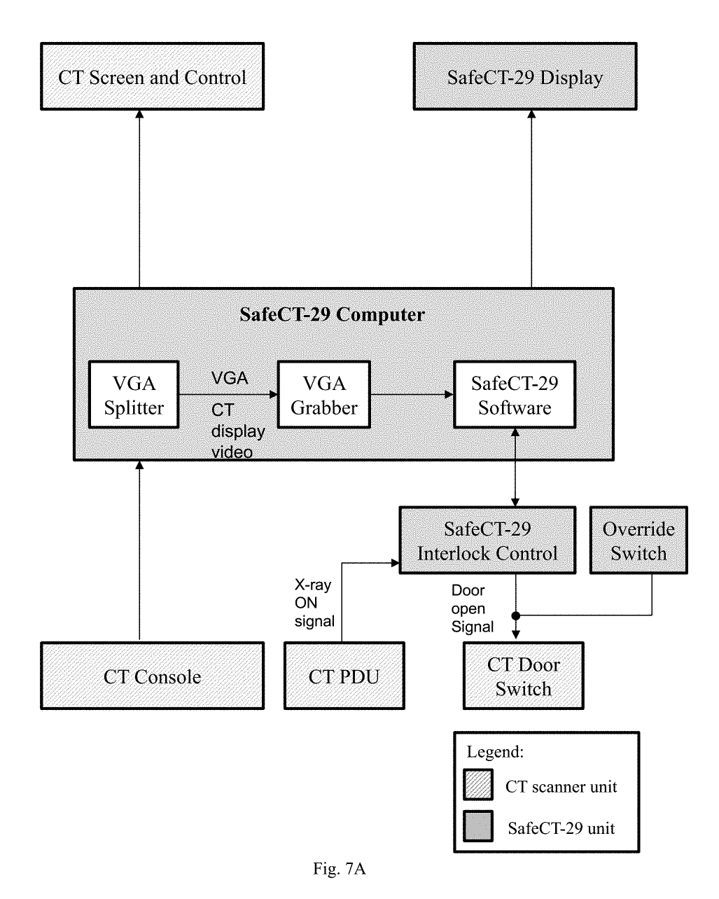

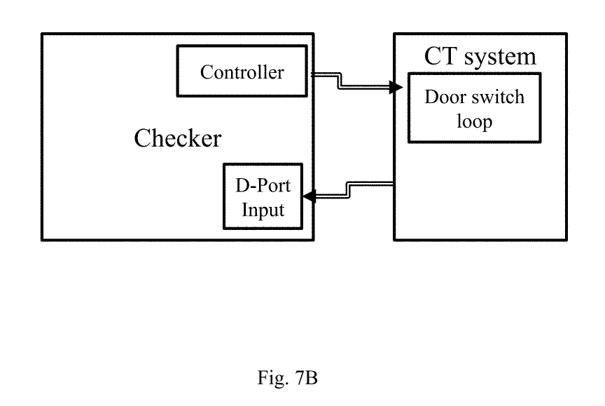

An exemplary SafeCT-29 and its components and subsystems is depicted in FIGS. 7A and 7B. The SafeCT-29 Computer may hosts a Video Splitter, a Video Grabber and the SafeCT-29 Software. The SafeCT-29 Computer may also manage the SafeCT-29 display and may control the SafeCT-29 Interlock Switch.

According to some embodiments, the Video splitter captures the CT Operator display video and sends a copy of same video signal to the Video Grabber. This function is performed continuously and automatically (the video splitter is always “on”; no user action or interface required). In fact, the Video Splitter is a passive device that takes the output signal out of the CT console and splits it into two identical output signals: one goes to the CT console and the other goes to the SafeCT-29 system.

According to some embodiments, the Video Grabber captures and converts an analog video signal, such as the signal produced by a CT Console to be displayed on the CT display, to digital video. The resulting digital data are computer files referred to as a video stream.

The SafeCT-29 Computer may include an OTS, high quality video grabber, which operates continuously and automatically (the video grabber is always “on”; no user action or interface required).

The rate of the grabber sampling is controlled by the SafeCT-29 software. When the software identifies that the CT is NOT in Scan or Preparation for scan mode, the sampling rate is lowered to prevent overheat and unnecessary power consumption.

The SafeCT-29 Display may be used to display the Dose Notification to the CT operator. It is a separate independent monitor (i.e., not the CT console display), that is “always on” (no “sleep mode”).

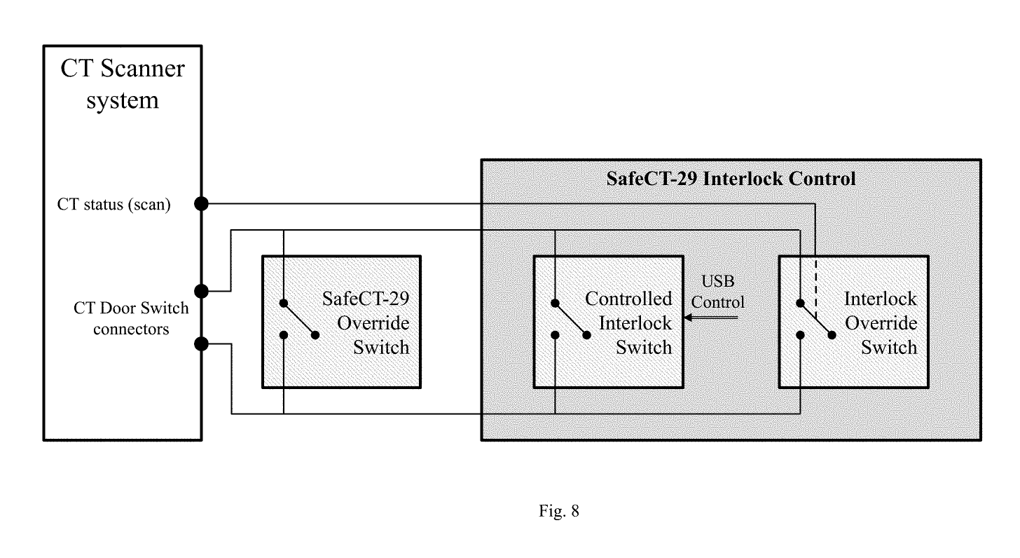

According to some embodiments, the SafeCT-29 may further include an Interlock Control comprising two controlled relays: Controlled Interlock Switch and Interlock Override Switch (FIGS. 7A and 8). The control relays prevent over-the-limit scans while ensuring that the SafeCT-29 system does not interfere with the scanner during a scan, even in case of malfunction or failure. The SafeCT-29 monitors the Interlock Control status to ensure proper operation and alerts the user in case of a failure.

According to some embodiments, the SafeCT-29 Interlock Control is connected to the related CT output port, following the CT Manufacturers' instructions. In conformance to the requirements of the 21CFR subchapter J, all CT systems include output ports for connecting door switches (FIGS. 7A and 7B) and X-Ray On warning lights and the connection instructions.

According to some embodiments, the checker controller is connected to the door switch loop of the CT System (FIG. 7B).

According to some embodiments, when the estimated dose level exceeds the predetermined threshold, a Notification or Alert is displayed on the SafeCT-29 Display, and the SafeCT-29 Computer generates a signal that opens the Interlock switch, thus preventing the scan.

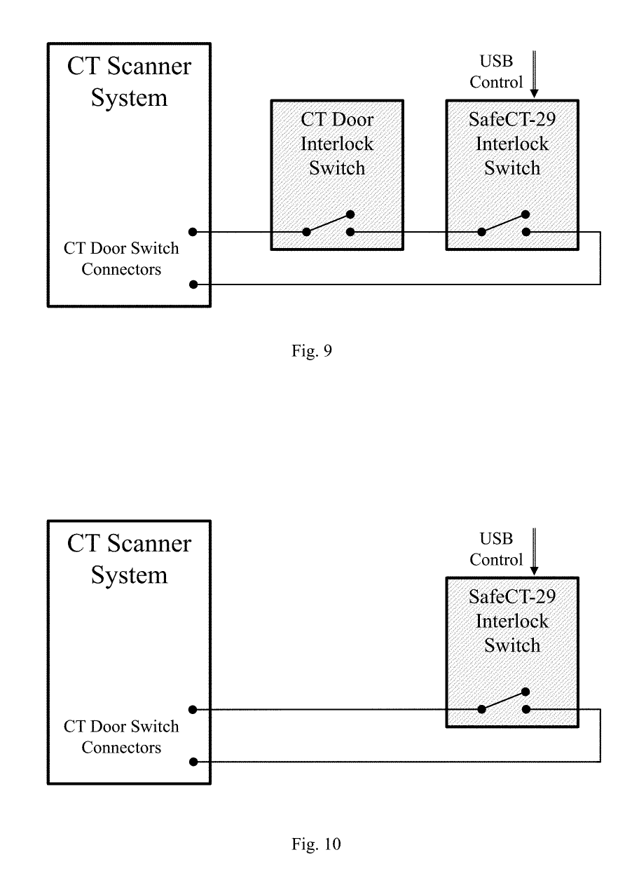

The Controlled Interlock Switch is a relay configured to stop a device upon the occurrence of certain events. CT systems contain a “Door Switch” which includes an interlock switch that stops the CT scan (or prevents initiating a new scan) when the CT room door is open. The SafeCT-29 Controlled Interlock Switch, which is controlled by the SafeCT-29 software (e.g., via USB connector), may be connected in series with the CT Door interlock switch (FIG. 9). In case a CT Door Switch is not installed at the site, the SafeCT-29 Interlock Switch may be connected directly to the door switch connectors CT System (FIG. 10).

Upon system initialization, the SafeCT-29 Computer may generate a signal (e.g., via the USB link) that sets the Controlled Interlock Switch so that scans can be performed. The switch opens the door switch loop by the SafeCT-29 Computer, when a Notification or Alert is displayed, thus preventing the operator from scanning in over-dose, as defined in XR-25 Standard.

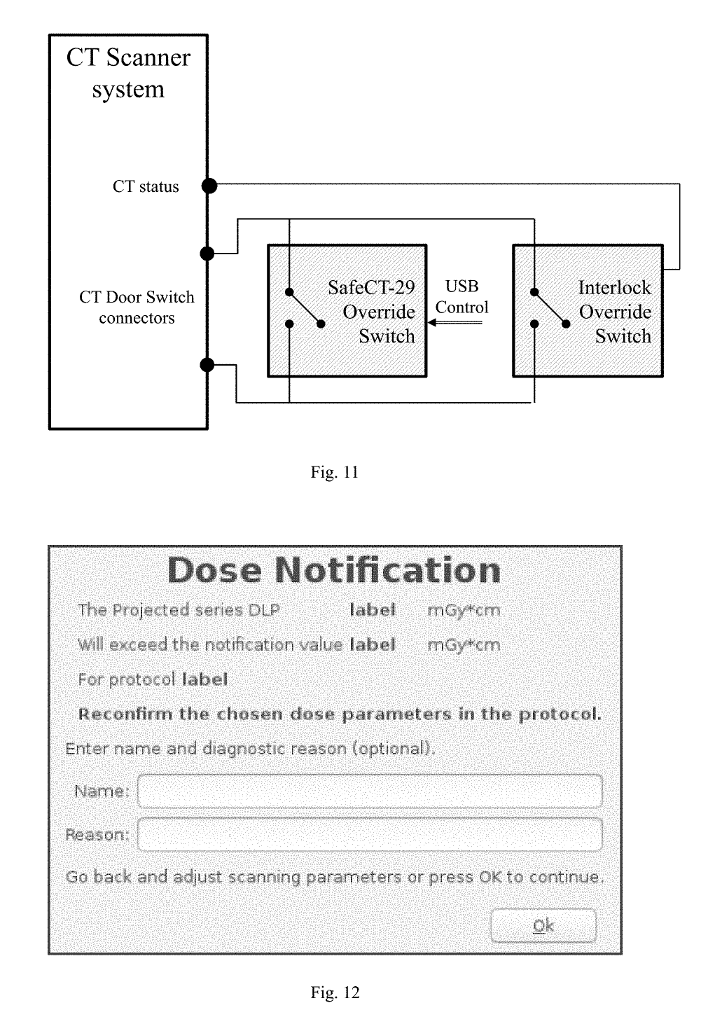

The Interlock Override switch prevents a potential accidental interruption during a scan, by the SafeCT-29 system. The Interlock Override switch, may be connected in parallel to the Controlled Interlock Switch (FIG. 11), is controlled by the CT X-ray ON warning signal (which, controls the X-Ray On lights). Whenever the X-Ray-on light signal is active (“Light On”), the Interlock Override switch is closed, thus overriding the entire SafeCT-29 system and preventing any accidental interruption during a scan.

The Interlock Override switch may be interfaced to the CT PDU (CT power distribution unit) conforming to the CT manufacturer's X-Ray light instructions.

According to some embodiments, an Override switch is implemented in the SafeCT-29 device (see, for example, FIGS. 7A and 8). The Override switch is configured to ensure that the CT scanner can be used (i.e. scans can be performed) in case of a SafeCT-29 failure. It is connected in parallel to the Controlled Interlock Switch. According to some embodiments, the Override Switch is a manually activated switch that is located in close proximity to the CT Operator. When activated, the Controlled Interlock Switch is bypassed and the scanner can be operated regularly.

According to some embodiments, the checker software system is intended for installation in a specified computer. According to some embodiments, the SafeCT-29 Software supports a variety of user interface and functionality features. According to some embodiments, the user operates the software via a standard (small footprint) keyboard, mouse, and the SafeCT-29 Display. According to some embodiments, the SafeCT-29 software includes a configuration file, in which commercial scanners and their software versions are defined, together with the related information on the location of the relevant information on the CT display and the scanner's workflow. The configuration file is continuously updated so that it includes the relevant data of any newly introduced CT scanner model.

According to some embodiments, notifications and permission requests may be provided.

### Example 3. Identifying CT Protocols and Dose Data

SafeCT-29 may include one or more configuration files, each includes information about which the location of the relevant information (e.g., radiation dose) on the CT system, such as the CT Display. Such file may also include information on the scanner's workflow. Relevant information may include dose data among other data. Information related to location may refer to position of the relevant information within the CT system, and/or within an image that the CT system produces.

Extracting dose data may be performed on an image, as follows: a video stream is received from the Video Grabber and analyzed continuously in real time. The CT Protocol, the estimated dose levels and exam ID (as appear on the CT display) are extracted.

According to some embodiments, identifying CT Protocol and dose data is performed by OTS OCR software, integrated with the SafeCT-29 Software. The integrated OCR software may preferably meet the following requirements:
a. may perform OCR reading in a time resolution which is sufficient for making a decision, to ensure that a scan cannot be initiated by the CT operator if the dose values exceed the Notification/Alert values;
b. may analyzes the picture repeatedly: data items are identified when the OCR output values are consistent, i.e. same results are received, consecutively, for multiple times;
c. may be capable to identify a protocol name in case of a difference of up to several characters (one or more) between the OCR output and a pre-defined list of protocols;
d. in the event that the SafeCT-29 software cannot identify a protocol, where the software includes a file of notifications and alerts, the software will search for a name of a group (e.g. “Head”) and, if found, will allocate the related notification and alert values; and
e. OCR accuracy: 99% for numeric values; 85% for text (OCR only). Accuracy of OCR and SafeCT-29 software, as defined in (b)-(e) above: for dose values (numeric data): 99.9% For Protocol data (alphanumeric): 98%.

### Example 4. Assessing Dose Level Relative to Thresholds

According to some embodiment, the system continuously compares the estimated dose levels and the protocol data that are extracted from the CT files or from the CT display video, using the OCR, to pre-defined Notification and Alert Values. In case the estimated dose exceeds the Notification Value or the Alert Value, the system performs the following actions:

a. A notification to the user is displayed on the SafeCT-29 Display

b. A command is generated to open the Interlock Switch (thus preventing the scan)

In case the system cannot match the Protocol or Protocol name with those defined in the Computer's internal database (for example, if a notification value was not set for the selected CT Protocol), a warning message is displayed to the user on the SafeCT-29 Display.

### Example 5. Notifications Display

Notifications, preferably directed to the CT operator, may be displayed on the SafeCT-29 Display (monitor). The SafeCT-29 software may generates the following notifications: Dose data, as extracted from the CT display by the OCR; Dose Notification, in case the estimated dose level is higher than the established Notification value, in accordance with the XR-25 Standard (FIG. 12); Dose Alert, in case the estimated accumulated dose level is higher than the established Alert value in accordance with the XR-25 Standard (FIG. 13); System status, failures and warnings; and the system is not required to display an alert if a corresponding Alert Value has not been set

### Example 6. System Operation Under Notifications and Notifications Removal

According to some embodiments, Dose Notification and Dose Alert may be removed under one or more of the following conditions: (1) the CT Operator changes the protocol's parameters so the estimated dose level does not exceed anymore the notification/alert value; (2) the CT Operator is identified and confirms the selected protocol's parameters. According to some embodiments, the CT Operator may further provide to the system reasoning for the selected protocol's parameters.

The software may generate an audio alert aimed to ensure that the user is aware of a notification, alert or warning that is displayed on the SafeCT-29 display. The audio alert may include at least one tone (“beep”) over a span of a few seconds (e.g. 2-6 seconds). The beep tone(s) may be generated by the computer internal speaker. The beep tone may be generated upon one or more of the following conditions: (i) when a Dose Notification or Dose Alert is displayed; and/or (ii) when the software identifies an error or failure that prevents scanning (e.g. Control Interlock Switch failure).

According to some embodiments, scanning is prevented by the system, upon display of a Notification or an Alert. Initially, upon startup, the software closes the Door switch loop. However, when an Alert is displayed on the SafeCT-29 display, the SafeCT-29 Computer may generate a signal that opens the Interlock switch, thus opening the door switch loop and preventing the initiation of a new scan. It should be noted however that neither Dose Notification nor Dose Alert interrupts a scan while in progress.

Thus, in normal operation, the checker-device maintains the CT door switch loop closed (i.e. enabling scanning). Checker-device opens the CT door switch loop when the estimated dose exceeds the pre-defined threshold(s).

Moreover, the checker-device software identifies that the scanner is in “Scan” mode (based on the information that is displayed on the CT Operator Display) and does not allow entering into “Alert Mode” or “Notification Mode” thus preventing potential accidental interruption.

Additionally, the checker-device Interlock Control includes an Interlock Override switch, which maintains the door switch closed during the scan, thus preventing any interruption by the checker-device system during a scan, even in case of a software error or failure.

The SafeCT-29 Display may have one or more of the following modes:
a. Normal operation, in which the identified CT Protocol (or Protocol Element) and the related dose levels are presented on the Display. No user action is required or associated with this mode.
b. Notification: a Dose Notification is displayed on the Display, requiring the user to validate CT dose data and reconfirm the CT Protocol parameters. The Notification disappears (and the display goes back to Normal Operation mode) when the dose levels are set below the Notification Value(s) or when the user confirm the scan parameters
c. Alert: a Dose Alert is displayed on the Display, requiring the user to validate CT dose data and reconfirm the CT Protocol parameters. The Dose Alert disappears (and the display goes back to Normal Operation mode) when the dose levels are set below the Alert Value(s) or when the user confirm the scan parameters
d. Failure: in case of a SafeCT-29, failure information is displayed on the Display. The user shall refer to the User Guide for assistance.

In case of a system failure, the user may disconnect the SafeCT-29 entirely by operating an Override Switch, and continue working with the CT scanning as if SafeCT-29 is not associated with the scanner. The Override Switch status may be displayed to the user.

The SafeCT-29 system may be designed to be “always on.” The system automatically enters its operational mode upon power up (no need for any user intervention such as login). The system may be powered-off or restarted through a software command or by powering off the SafeCT-29 PC.

Additionally, while the CT is in operation, the SafeCT-29 display continuously displays any relevant information (i.e. not in “sleep” mode).

### Example 7. Auto Scanner Model Identification and Calibration Process for SafeCT-29

According to some embodiments, SafeCT-29 system is connected as an add-on to an existing CT Operator display, wherein the Video splitter captures the CT Operator display video and sends a copy of same video signal to the Video Grabber, to be analyzed by an integrated OCR software to detect the radiation parameter values from the analyzed imagery. The layout of data parameters, including the location of the radiation parameters on the CT Operator display (i.e. within the internal-display signal) can vary from one CT model to another.

According to some embodiments, a periodically updated database of CT models is defined and stored in the SafeCT-29 system memory. The Video splitter captures the CT Operator display video and sends a copy of same video signal to the Video Grabber, wherein said captured image is compared to the database of CT models, optionally associated with specific software versions and their screen layouts. The identification process is presented in FIGS. 16-19.

According to some embodiments, at the end of the calibration and optimization process, a system's user can perform a Quality Control (QC) by reviewing the results, which can be visualized through a Graphical User Interface (GUI) such as the exemplary layout provided in FIG. 19, and validate the results presented, for example, by clicking an approval button. Alternatively, the user can perform a manual calibration of some of the presented characteristics, such as X, Y coordinates, width, height, zoom level and threshold function.

The complete process or a portion of the process, such as manual QC, can be re-performed and updated periodically or according to any requirements set by a system's user.

1. A method of identifying and calibrating CT model display layout, comprising:
providing a database of CT models comprising a plurality of CT models each associated with at least one unique element and at least one screen layout comprising at least screen elements, a video splitter configured to capture images of a CT operator display of an existing CT system, and a video grabber,
connecting the video splitter and the video grabber to the CT operator display,
capturing at least one image of the CT operator display;
transmitting the at least one captured image to the video grabber;
defining a first search window for scanning throughout the captured image;
identifying a CT model by comparing the contents of the first search window with the at least one unique element in each CT model within the database of CT models;
retrieving the at least one screen element associated with the identified CT model;
generating a screen layout comprising the at least one screen element; and
calibrating and optimizing scan parameters for each screen element.

2. The method according to claim 1, wherein the database further comprises at least one software version for at least one CT model.

3. The method according to claim 2, wherein the identifying a CT model further comprises identification of the software version of the CT model.

4. The method according to claim 1, further comprising changing the size of the first search window if a unique element was not identified.

5. The method according to claim 1, wherein generating a screen layout further comprises:
defining a second search window for scanning throughout the captured image;
comparing contents of the second search windows with screen elements;
validating values of the screen elements; and
verifying that all screen elements comply with preset rules.

6. The method according to claim 5, further comprising changing the size of the second search window if a unique element was not identified.

7. The method according to claim 1, wherein the calibrating and optimizing scan parameters comprises:
defining a range of zoom levels;
defining a range of threshold functions;
executing OCR functionality for each zoom level in the range of zoom levels and for each threshold function for the range of threshold functions;
identifying a correct value as the most common value of the series of OCR executions; and
identifying the fastest OCR execution time for all correct values.

8. The method according to claim 7, wherein all steps are executed for all screen parameters.

9. The method according to claim 8, further comprising:
storing data comprising: x coordinate, y coordinate, width, height, zoom level, threshold function, OCR execution time and correct value, for all screen parameters, and
presenting a graphic representation of the stored data.

10. The method according to claim 9, further comprising performing manual quality control.

11. The method according to claim 10, wherein the performing manual quality control comprises manual approval of the presented stored data.

12. The method according to claim 10, wherein the performing manual quality control comprises manual update of a portion of stored data, comprising: x coordinate, y coordinate, width, height, and zoom level.

13. A method of calibrating and optimizing scan parameters, comprising:
providing a scanned image;
identifying screen elements in the scanned image;
defining a range of zoom levels;
defining a range of threshold functions;
executing OCR functionality for each zoom level in the range of zoom levels and for each threshold function for the range of threshold functions;
identifying a correct value as the most common value of the series of OCR executions; and
identifying the fastest OCR execution time for all correct values.

14. A method of identifying and calibrating CT model display layout, comprising:
providing a database of CT models comprising a plurality of CT models each associated with at least one unique element and at least one screen layout comprising a list of screen elements, a video splitter configured to capture images from a CT operator display of an existing CT-system, a video grabber, and a CT checker device, the CT checker device comprising:
an input port configured to be associated with the existing CT-system, the input port configured to obtain scanning data and provide corresponding signal;
processing circuitry; and
a control unit, connected to a control switch within the existing CT system,
connecting the video splitter and the video grabber to the CT operator display;
capturing at least one image of the CT operator display;
transmitting the at least one captured image to the video grabber;
defining a first search window for scanning throughout the captured image;
identifying a CT model by comparing the contents of the first search window with the at least one unique element in each CT model within the database of CT models;
retrieving the at least one screen element associated with the identified CT model; and
generating a screen layout comprising the at least one screen element; and
calibrating and optimizing scan parameters for each screen element,
wherein the processing circuitry is configured to obtain the screen elements; detect a radiation parameter value from the scanning data; compare the detected radiation parameter value with a predetermined threshold; and generate an operation-signal based on the comparison,
and wherein the control unit is configured affect an operation of the existing CT system, based on the operation-signal, by toggling the state of the control switch thereby preventing radiation, wherein the checker is separate from the existing CT system.
